(19) United States
(12) Patent Application Publication
(10) Pub. No.: US 2012/0009272 A1
(43) Pub. Date: Jan. 12, 2012

(54) Compositions and methods for creating an air release mechanism to maintain skin health

(75) Inventor: Jennifer F. Bartels, Hammond, LA (US)

(73) Assignee: Penguin IP Holdings Inc.

(21) Appl. No.: 13/222,668

(22) Filed: Aug. 31, 2011

Related U.S. Application Data
(63) Continuation of application No. 10/369,368, filed on Feb. 18, 2003.
(60) Provisional application No. 60/358,330, filed on Feb. 19, 2002.

(51) Int. Cl. A61K 8/19; A61Q 19/00; A61K 8/97

(52) U.S. Cl. 424/600; 424/769

(57) ABSTRACT

A topical composition and method for the treatment of the symptoms of diaper rashes and skin irritations by creating an air circulating mechanism comprising a) bicarbonate of soda; b) corn starch; c) citric acid; d)a carrier oil; e) a pharmaceutically acceptable moisturizer; f) fragrance oil; and g) food grade coloring. The carrier oil is selected from the group consisting of almond oil, coconut oil, sunflower oil, apricot kernel oil, avocado oil, canola oil, evening primrose oil, grapeseed oil, hazelnut oil, jojoba oil, macadamia oil, olive oil, sesame oil, rosehip seed oil, and mixtures thereof. The pharmaceutically acceptable moisturizer is either purified water or witch hazel. In its preferred embodiments, the composition can result in a loose powder for application directly to the diapered skin, an effervescent bath tablet, and as an additive to the bodyfacing layer of a diaper.

### BACKGROUND OF THE INVENTION

The present invention relates generally to compositions and methods to treat a skin condition commonly known as “diaper rash” by creating an air release mechanism to maintain skin health.

A skin condition commonly known as diaper rash is prevalent among infants everywhere. It has been acknowledged that skin preparations cover a range of uses—from protecting the skin against inclement weather, irritations, and in the case of infants, diaper rash. It has also been acknowledged that there is a wide range of patented formulations, each dealing with skin health, irritations, and conditions in different ways.

Bacterial and microbial growth has been addressed by Ashley in U.S. Pat. No. 5,762,945. Barrier creams have been formulated in a number of patents, including by Enjolias in U.S. Pat. No. 5,091,193 and by Pichierri in U.S. Pat. No. 5,194,261.

The present invention seeks to alleviate skin conditions, primarily infant diaper rash, by the mechanism of air release in a certain compound, heretofore known as sodium bicarbonate (i.e., baking soda, or sodium hydrogen carbonate.) The properties of sodium bicarbonate, as they relate to the present invention, are as follows:

1) in an aqueous solution, sodium bicarbonate begins to release CO2 at room temperature

2) when dry, sodium bicarbonate begins to release CO2 when either heated or combined with an acid, such as citric acid.

A prior art reference was found in U.S. Pat. No. 5,306,487 issued to Karapasha, which discusses a high capacity composition which contains members of the activated carbon family. However, this patent uses an entirely different composition and claims only to address odor control, not air release.

While sodium bicarbonate has been used in diaper assembly as an odor control composition, prior patents such as U.S. Pat. No. 6,518,479 issued to Kimberly Clark have placed the composition within the underlayers of absorbent materials via a meltblown method. Two factors in this process do not help to solve the present invention's goal of air release:

1) in the manufacture of the diaper and of the process of putting the bicarbonate composition into the diaper, there is a heat process which causes at least some of the CO2 to be released before the infant ever wears the diaper; and

2) the sodium bicarbonate composition is trapped in the underlayers of the diaper, where urine is wicked away to, so there is no contact of the sodium bicarbonate to the skin. Actually, the prior art works exactly as the inventor intends, since that specific goal as disclosed in the patent document is to move moisture, primarily urine, away from the skin and combine it effectively with the bicarbonate composition to neutralize the odor. The baby is left with a slightly moist bottom and a generally wet environment for the diapered skin.

Because of the above 2 facts, the benefit of the bicarbonate of soda composition is limited to odor control as it relates to U.S. Pat. No. 6,518,479 issued to Kimberly Clark.

Other methods of dispersing air (CO2) circulation to the skin as it relates to diaper rash can be derived from this composition. One is an effervescent tablet for the infant's bath. Effervescent tablets are just what the name indicates: when met with an aqueous solution, such as water, as in the bath, combined with citric add in the tablet, the tablet will release all of its CO2 into the bath water as the baby sits in the bath, which is what causes the effervescing or bubbling action. It is known from homeopathic and years of childcare experience and also acknowledged by pediatricians worldwide that there is no substitute for water and fresh air in alleviating the uncomfortable effects of diaper rash. The bath treatment is also a pleasant and fun experience for infant and caregiver.

Another embodiment of the composition is a loose powder which can safely be sprinkled onto the skin by the caregiver after the bath or at each diapering session. This composition and method would further serve to increase air circulation in and around the skin area within the diaper and would begin to release CO2 when met with wetness and/or an acid as in an add stool.

Moreover, in the case of the diaper, the composition would be applied to the bodyside layer either

1) after the diaper is manufactured

2) at or after the manufacture of said bodyside layer provided there is no heat process used.

3) during manufacture of the diaper, as long is there is no heat process sufficient to release significant amounts of CO2 from the composition.

This composition would be applied using techniques which are known in the arts.

Consequently, a need has been felt for providing a composition and method for treating diaper rash by releasing air and increasing air circulation on and around the skin area as it relates to infant diaper rash.

Moreover, the present invention is unique and innovative due to its composition and methods and its difference in approach to the prior art in treating diaper rash.

### SUMMARY OF THE INVENTION

It is therefore an object of the present invention to provide an innovative and effective composition and methods for treating diaper rash.

It is yet another object of the present invention to provide another formulation and method besides barrier creams and pH adjusting compositions to treat diaper rash and skin irritations.

It is a feature of the present invention to provide a navel use for the formulation in the form of a loose powder, effervescent tablet for the bath, and an additive for the outer bodyside layer (closest to the skin) of a diaper.

Advantages of the present invention are that it is easily applied, the ingredients are readily available and inexpensive to manufacture, the active ingredients have been proven to be safe over years of uses in other applications, and the composition is effective by itself or in combination with other conventional treatments.

### DESCRIPTION OF THE PREFERRED EMBODIMENTS

It has now been discovered that a composition of a combination of sodium bicarbonate, corn starch, citric acid, a light oil, a pharmaceutically acceptable moisturizer, and a fragrance will act with either a bath or with bodily functions to increase air circulation on and around the skin to alleviate diaper rash.

### Example 1

In its preferred embodiment, the composition is made of the following ingredients:

a. 5-80% sodium hydrogen carbonate (bicarbonate of soda)

b. 5-80% cornstarch

c. 5-50% citric acid

d. 0.5-30% a carrier oil

e. 0.001-2% pharmaceutically acceptable moisturizer

f. 0.001-5% fragrance oil

g. 0.001-2% food grade coloring

wherein said pharmaceutically acceptable moisturizer is purified water or witch hazel, and where carrier oil is selected from the group consisting of almond oil, coconut oil, sunflower oil, apricot kernel oil, avocado oil, canola oil, evening primrose oil, grapeseed oil, hazelnut oil, jojoba oil, macadamia oil, olive oil, sesame oil, rosehip seed ail, and mixtures thereof.

### Example 2

In a particularly preferred embodiment for the effervescent tablet, the composition is made of the following ingredients:

a. 56.7 grams sodium hydrogen carbonate (bicarbonate of soda)

b. 56.7 grams corn starch

c. 28.35 grams citric acid

d. 21.26 grams carrier oil

e. 3.825 grams pharmaceutically acceptable moisturizer

f. 1.275 grams fragrance oil

g. 1.275 grams food grade coloring

wherein said pharmaceutically acceptable moisturizer is purified water or witch hazel, and where carrier oil is selected from the group consisting of almond oil, coconut oil, sunflower oil, apricot kernel oil, avocado oil, canola oil, evening primrose oil, grapeseed oil, hazelnut oil, jojoba oil, macadamia oil, olive oil, sesame oil, rosehip seed ail, and mixtures thereof.

### Example 3

In a particularly preferred embodiment for the loose powder, the composition is made of the following ingredients:

a. 113.4 grams sodium hydrogen carbonate (bicarbonate of soda)

b. 113.4 grains corn starch

c. 1.275 grams fragrance oil

### Example 4

In a particularly preferred embodiment for the diaper additive, the composition is made of the following ingredients:

a. 113.4 grams sodium hydrogen carbonate (bicarbonate of soda)

b. 113.4 grams corn starch

c. 5 grams carrier oil

d. 1.275 grams fragrance oil

wherein carrier oil is selected from the group consisting of almond oil, coconut oil, sunflower oil, apricot kernel oil, avocado oil, canola oil, evening primrose oil, grapeseed oil, hazelnut oil, jojoba oil, macadamia oil, olive oil, sesame oil, rosehip seed oil, and mixtures thereof. The addition of a carrier oil might serve to make the particles adhere to the skin more easily and reduce airborne particles.

The foregoing descriptions are included to illustrate the operation of the preferred embodiment and is not meant to limit the scope of the invention. From the foregoing description, many variations will be apparent to those skilled in the art that would yet be encompassed by the spirit and scope of the invention.

1. A composition for topically treating diaper rash and other skin irritations by creating an air releasing and circulating mechanism comprising a) bicarbonate of soda; b) corn starch; c) citric acid; d)a carrier oil; e) a pharmaceutically acceptable moisturizer, f) fragrance oil; and g) food grade coloring.

2. The composition of claim 1, wherein said pharmaceutically acceptable moisturizer is purified water or witch hazel.

3. The composition of claim 1, wherein carrier oil is selected from the group consisting of almond oil, coconut oil, sunflower oil, apricot kernel oil, avocado oil, canola oil, evening primrose oil, grapeseed oil, hazelnut oil, jojoba oil, macadamia oil, olive oil, sesame oil, rosehip seed oil, and mixtures thereof.

4. The composition of claim 1, mixed and formed using techniques known in the arts.

5. The composition of claim 1, further comprising calcium carbonate

6. A composition for topically treating diaper rash and skin irritations comprising:
a. 5-80% sodium hydrogen carbonate (bicarbonate of soda)
b. 5-80% cornstarch
c. 5-50% citric acid
d. 0.5-30% a carrier oil
e. 0.001-2% pharmaceutically acceptable moisturizer
f. 0.001-5% fragrance oil
g. 0.001-2% food grade coloring
wherein said pharmaceutically acceptable moisturizer is purified water or witch hazel, and where carrier oil is selected from the group consisting of almond oil, coconut oil, sunflower oil, apricot kernel oil, avocado oil, canola oil, evening primrose oil, grapeseed oil, hazelnut oil, jojoba oil, macadamia oil, olive oil, sesame oil, rosehip seed oil, and mixtures thereof.

7. The composition of claim 6, formulated as a loose powder, additive for the bodyfacing layer of an absorbent article, and an effervescent bath tablet.

8. The composition of claim 6, wherein said absorbent article is a diaper.

9. A method for treating diaper rash and skin irritations in a patient by creating an air releasing and circulating mechanism to be either sprinkled on the diaper area of the patient's skin, added to the patient's bath water, or as an additive for the bodyfacing layer of the patient's absorbent article a composition comprising a) bicarbonate of soda; b) corn starch; c) citric acid; d)a carrier oil; e) a pharmaceutically acceptable moisturizer; f) fragrance oil; and g) food grade coloring

9. The method of claim 9 wherein said patient's absorbent article is a diaper.

10. The method of claim 9 wherein said carrier oil is selected from the group consisting of almond oil, coconut oil, sunflower oil, apricot kernel oil, avocado oil, canola oil, evening primrose oil, grapeseed oil, hazelnut oil, jojoba oil, macadamia oil, dive oil, sesame oil, rosehip seed oil, and mixtures thereof.

11. The method of claim 9 wherein said pharmaceutically acceptable moisturizer is either purified water or witch haze.

12. The method of claim 9, further comprising calcium carbonate.
